(19) United States
(12) Patent Application Publication
(10) Pub. No.: US 2023/0178249 A1
(43) Pub. Date: Jun. 8, 2023

(54) HEALTH LEVEL DETERMINATION SYSTEM, HEALTH LEVEL DETERMINATION PROGRAM, AND HEALTH LEVEL DETERMINATION SERVER

(71) Applicant: TANITA CORPORATION, Tokyo (JP)

(72) Inventors: Miyuki KODAMA, Tokyo (JP); Hisako TANAKA, Tokyo (JP); Masanori IKEDA, Tokyo (JP)

(21) Appl. No.: 18/161,153

(22) Filed: Jan. 30, 2023

Related U.S. Application Data
(63) Continuation of application No. PCT/JP21/27943, filed on Jul. 28, 2021.

(30) Foreign Application Priority Data
Jul. 31, 2020 (JP) 2020-130946

(51) Int. Cl. G16H 50/30; A61B 5/053; G16H 10/60

(52) U.S. Cl. CPC G16H 50/30; A61B 5/053; G16H 10/60

(57) ABSTRACT

A system for determining the health level of a user is provided. The health level determination system (520) includes a bioelectrical impedance acquisition section (53) that acquires bioelectrical impedances of a plurality of body parts of a user, a body composition estimation section (54) that estimates the body composition of a plurality of items of the user from the bioelectrical impedances of the plurality of body parts, and a body composition estimation section (54) that estimates the body composition of a plurality of items of the user based on the body composition of the plurality of items, at least partially, indirectly, or directly. The body composition estimation section (54) estimates the plurality of body compositions of the user from the bioelectrical impedances of the plurality of body parts.

### CROSS REFERENCE TO RELATED APPLICATION

The present application claims priority to JP Application No. 2020-130946 filed Jul. 31, 2020, the entire contents of which are hereby incorporated by reference.

### FIELD

The present disclosure relates to a health level determination system, a health level determination program, and a health level determination server for determining the health level of a user.

### BACKGROUND

In general, medical checkups include measurements of height, weight, chest circumference, chest X-ray, urine protein, urine occult blood, GOT, GPT, LDL-C, HDL-C, TG, uric acid level, HbA1c, FPG, hematocrit, RBC, CRP, maximum blood pressure, minimum blood pressure, etc. Based on the results of these medical checkups, a physician can advise the examinee on the health level or health risk. In addition, a method has been proposed to determine the health level or health risk using a predetermined formula based on the results of medical checkups (see JP 2002-063278 A).

### SUMMARY

However, medical checkups require examinees to visit a medical institution to undergo tests, and are generally performed only once a year. It is advantageous to make those who are living a life that increases their health risks (decreases their health) aware of those risks in a shorter period of time, while it is also advantageous to show the results in a shorter period of time to those who are living a life that lowers their health risks (increases their health).

Therefore, it is an object of the present disclosure to provide a system that can determine the health level in a simple manner.

Since health level and health risk are concepts that are inversely related, determining the health level means determining the health risk. In this disclosure, the concept of health level is used to describe embodiments, but the replacement of health level with health risk is also within the scope of the disclosure. The concept of the health risk includes the possibility of suffering from a disease.

A health level determination system of one aspect of the present disclosure comprising: a bioelectrical impedance acquisition section that acquires one or more bioelectrical impedance of one or more body parts of a user; and a health level determination section that determines a health level of the user based at least partially, indirectly or directly, on the bioelectrical impedance of the one or more body parts.

Since body composition analyzers, which are widespread in general households, measure the bioelectrical impedance of one or more body parts of the user, this configuration allows the user to make a simple determination of the health level using, for example, a body composition analyzer at home.

The above health level determination system may further comprise a body composition estimation section that estimates a plurality of body compositions of the user from the bioelectrical impedances of the one or more body parts, and the health level determination section may determine the health level of the user based, at least partially, indirectly or directly, on the plurality of body compositions.

The above health level determination system may further comprise a medical checkups estimation section that estimates the plurality of medical checkup results of the user from the plurality of body compositions, and the health level determination section may determine the health level of the user based at least partially on the plurality of medical checkup results. The medical checkup result estimation section may estimate the plurality of medical checkup results of the user from the plurality of body compositions using an inference model (multiple regression analysis, principal component analysis, decision tree, neural network, Bayesian network, etc.). The health level determination section may determine the health level of the user based at least partially on the plurality of medical checkup results using an inference model (multiple regression analysis, principal component analysis, decision tree, neural network, Bayesian network, etc.).

In the above health level determination system, each of the plurality of medical checkup results may have a significant correlation with the plurality of body compositions. Here, the significant correlation means that the correlation is statically significant. In other words, there is a correlation that is statistically or probabilistically unlikely to be a coincidence and is considered to be meaningful.

In the above health level determination system, the health level determination section may determine whether the user is healthy, unhealthy, or indeterminate.

The health level determination system described above may further comprise an additional information acquisition section that requests an input of additional information that was not used to determine the health level when the result of the determination is indeterminate, and the health level determination section may further determine the health level using the additional information.

The above health level determination system may further comprise an output section that outputs the result of the determination of the health level, and the output section may display the result of the determination by pointing to the result of the determination on a clock-like graphic.

In the above health determination system, the output section may point to a predicted future determination result on the clock-like graphic.

A health level determination program of one aspect of the present disclosure, when executed by a processor, has a configuration that make the processor function as a health level determination section that determines a health level of a user based, at least partially, indirectly or directly, on bioelectrical impedances obtained from one or more body parts of the user. The health level determination program is configured to make the processor receive one or more bioelectrical impedance of one or more body parts of a user.

A health level determination server of one aspect of the present disclosure has a configuration having a health level determination section that determines a health level of a user based, at least partially, indirectly or directly, on bioelectrical impedances obtained from one or more body parts of the user. The health level determination sever is configured to receive one or more bioelectrical impedance of one or more body parts of a user. Further, a health level determination method of a further aspects of the present disclosure comprises acquiring, by means of a bioelectrical impedance acquisition section, one or more bioelectrical impedance of one or more body parts of a user; and determining, by means a health level determination section, a health level of the user based at least partially, indirectly or directly, on the bioelectrical impedance of the one or more body parts.

### BRIEF DESCRIPTION OF DRAWINGS

FIG. 1 shows a schematic diagram of the external configuration of a health level determination system according to the present disclosure;

FIG. 2 shows the usage of the measurement device according to a first embodiment of the present disclosure;

FIG. 3 shows a block diagram showing the hardware configuration of the health level determination system according to the first embodiment of the present disclosure;

FIG. 4 shows a block diagram showing the functional configuration of the health level determination system according to the first embodiment of the present disclosure;

FIG. 5 shows a correlation between HDL cholesterol (vertical axis), which is a biochemical test value, and fat mass (kg) (horizontal axis), which is body composition information, according to the first embodiment of the present disclosure;

FIG. 6 shows a correlation between HDL cholesterol (vertical axis), which is a biochemical test value, and the amount of visceral fat (cm²) (horizontal axis), which is body composition information according to the first embodiment of the present disclosure;

FIG. 7 shows a graph showing an accuracy of the estimation using plural kinds of body composition information according to the first embodiment of the present disclosure;

FIG. 8 shows a block diagram of the functional configuration of the health level determination system according to a second embodiment of the present disclosure;

FIG. 9 shows a graph showing an accuracy of determining the health level using the multi-item body composition information for the first and second embodiments of the present disclosure;

FIG. 10A shows a graph for explaining an accuracy of determining the health level using the plurality of body composition information according to the first and second embodiments of the present disclosure;

FIG. 10B shows a graph for explaining an accuracy of determining the health level using the plurality of body composition information according to the first and second embodiments of the present disclosure;

FIG. 11 shows an example of a health advice screen according to the embodiment of the present disclosure;

FIG. 12 shows another example of a health advice screen according to the embodiment of the present disclosure; and

FIG. 13 shows a block diagram of a health level determination system according to the third embodiment of the present disclosure.

### EMBODIMENTS OF THE DISCLOSURE

The embodiments of the present disclosure are described below with reference to the drawings. The embodiments described below are examples of how the disclosure may be implemented, and do not limit the disclosure to the specific configuration described below. In implementing the present disclosure, specific configurations may be adopted as appropriate according to the embodiments.

The health level estimation system of the embodiments described below measures one or more bioelectrical impedances of one or more body parts of a user, and determines the health level of the user based at least partially, indirectly or directly, on the measured bioelectrical impedances of the one or more body parts. The method for determining the health level based indirectly on the bioelectrical impedance includes: (1) a method for estimating the body composition of a plurality of items from the bioelectrical impedances of one or more body parts and determining the health level based on the estimated plurality of body compositions; and (2) a method for estimating the body composition of a plurality of items from the bioelectrical impedances of one or more body parts, estimating medical checkup results of multiple items from the estimated body composition of multiple items, and determining the health level based on the estimated medical checkup results of multiple items.

No matter whether the method is to determine the health level by estimating the medical checkup results or by determining the health level without estimating the medical checkup results, even though it is desirable to base the determination on multiple items of body composition (for example, different kinds of items of the body compositions such as fat mass and muscle mass, or items of the body compositions of different body parts such as whole body and right leg), it may also be based on a single item of body composition and at least one item of the user's biometric information (height, age, gender, and weight) described below.

In the following, the method of estimating the body composition from the bioelectrical impedance, further estimating the medical checkup result from the body composition, and determining the health level from the medical checkup result is described as the first embodiment, and the method of estimating the body composition from the bioelectrical impedance and determining the health level from the body composition without estimating the medical checkup result is described as the second embodiment, the method of directly determining the health level from bioelectrical impedance without estimating body composition and medical checkup results is explained as the second form of implementation.

In any of the embodiments, since health risks are determined from the information in the body (i.e. bioelectrical impedance) that shows continuous lifestyle habits, compared to determination of health risk based on medical checkups, which cannot be performed frequently because of the burden on users and medical institutions, or determination of health risk based on genetic testing services, which can be tested only by sending saliva, etc., but can only be evaluated based on congenital perspective, the system of the embodiment allows users to determine their health risks in their daily lives with less burden and taking their lifestyle into consideration. Not only is the burden less, but it is also assumed that there are cases in which health risks can be more accurately determined by using the system of the embodiment, which can measure and track changes every day, than by determining health risks based only on medical checkups, which is taken only once a year and cannot be said to represent the daily state.

### First Embodiment

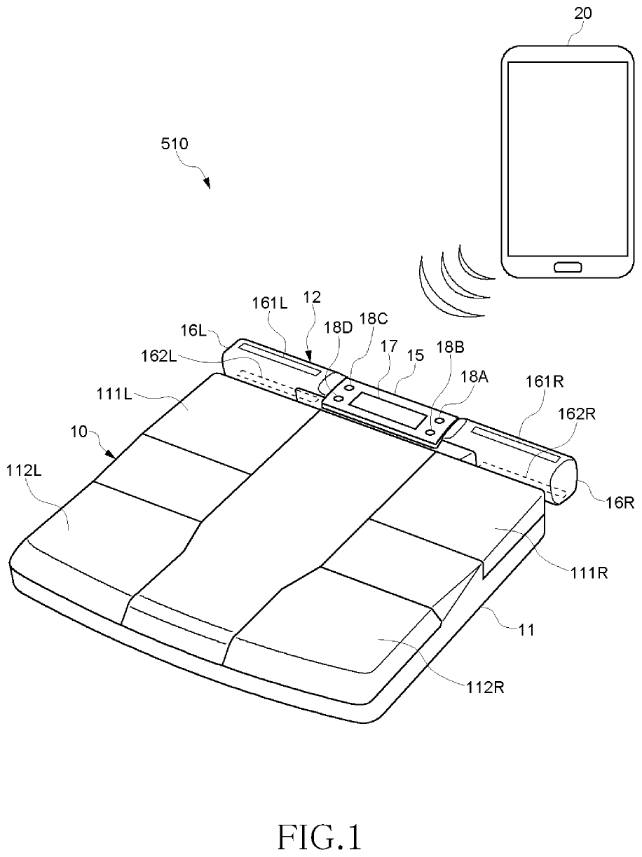

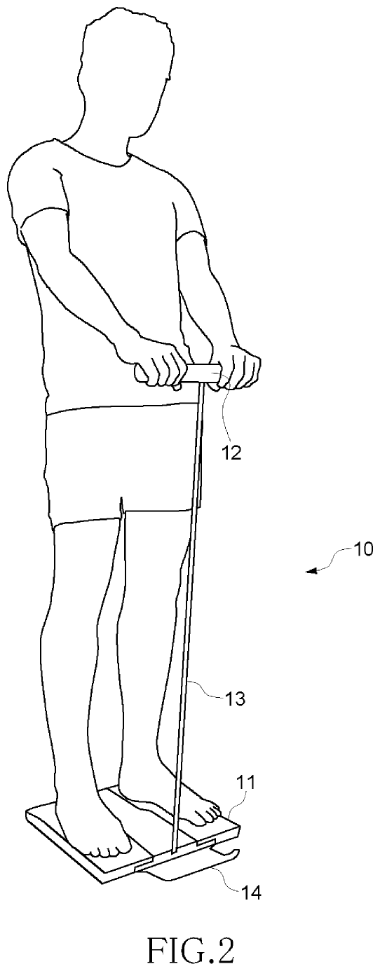

FIG. 1 shows a schematic diagram of the external configuration of a health level determination system according to the present disclosure. FIG. 2 shows the usage of the measurement device according to a first embodiment of the present disclosure. In this embodiment, the health level determination system 510 comprises a measurement device 10 and an information processing terminal (hereinafter referred to as “user terminal”). The measurement device 10 is a body composition analyzer that can measure body composition such as body fat by measuring a potential difference caused by a weak electric current flowing in the body, applying the measurement principle of Bioelectrical Impedance Analysis (BIA).

The measurement device 10 has a main unit 11 and a handle unit 12. The main unit 11 and the handle unit 12 are electrically connected by a connection cord 13. The handle unit 12 can be accommodated in a housing section 14 provided in the main unit 11. When the handle unit 12 is accommodated in the accommodating section 14, the connection cord 13 is wound up by a winding mechanism not shown inside the main unit 11 and accommodated inside the main unit 11.

The main unit 11 is equipped with an energizing electrode 111R and a measuring electrode 112R for the right foot on the right side of the upper surface, and an energizing electrode 111L and a measuring electrode 112L for the left foot on the left side of the upper surface.

The handle unit 12 has an approximate bar shape, with a handle body 15 in the center of it, and grips 16R and 16L on both sides of the handle body 15. The handle body 15 is provided with a display panel 17 and operation buttons 18A to 18D. The grip 16R is provided with an energizing electrode 161R and a measuring electrode 162R for the right hand, and the grip 16L is provided with an energizing electrode 161L and a measuring electrode 162L for the left hand.

In the manner of use shown in FIG. 2, the user can measure his/her body composition by standing upright on the main unit 11 with bare feet and holding the handle unit 12 with both hands with both arms extended. At this time, the base of the right toe contacts the energizing electrode 111R, the heel of the right foot contacts the measuring electrode 112R, the base of the left toe contacts the energizing electrode 111L, the heel of the left foot contacts the measuring electrode 112L, the fingers of the right hand contact the energizing electrode 161R, the palm of the right hand contacts the measuring electrode 162R, the fingers of the left hand contact the energizing electrodes 161L, and the palm of the left hand contacts the measuring electrode 162L.

The main unit 11 is also equipped inside with a load cell for measuring the weight. The weight of the user on the main unit 11 as shown in FIG. 2 can be measured. The load cell consists of a straining body of a metal member that deforms in response to the load and a strain gauge that is affixed to the straining body. When a user rides on top of the measurement device 10, the load of the user causes the straining body of the load cell flexes and the strain gauge expands or contracts. The resistance value (output value) of the strain gauge changes in accordance with the expansion or contraction. The measurement device 10 calculates the weight from the difference between the output value of the load cell when no load is applied (zero point) and the output value when a load is applied. The configuration regarding the measurement of weight using the load cell can be the same as that used in general scales.

The user terminal 20 is a portable terminal equipped with a processor capable of executing application programs, a memory for temporarily storing data necessary for processor processing, an internal storage such as flash memory for storing application programs and generated data, a touch panel, and various interfaces, etc. The user terminal 20 is also equipped with a wireless communication device for connecting to the Internet and a short-range communication device for connecting to other nearby devices. The measurement device 10 is equipped with a short-range communication device for connecting to other nearby devices. The measurement device 10 and the user terminal 20 can send and receive various types of information through short-range wireless communication (e.g., Bluetooth (registered trademark)) by pairing with each other.

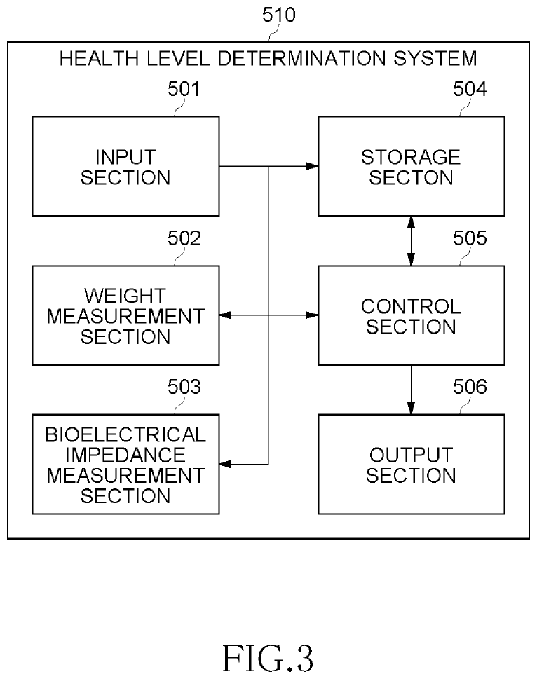

FIG. 3 shows a block diagram showing the hardware configuration of the health level determination system 510 according to the first embodiment of the present disclosure. The health level determination system 510 has an input section 501, a weight measurement section 502, a bioelectrical impedance measurement section 503, a memory section 504, a control section 505, and an output section 506. Of these components, the weight measurement section 502 and the bioelectrical impedance measurement section 503 are provided in the measurement device 10. However, since the measurement device 10 and the user terminal 20 can communicate with each other as described above, the input section 501, the storage section 504, the control section 505, and the output section 506 may be provided in either the measurement device 10 or the user terminal 20, or both. In this embodiment, as an example, the weight measurement section 502 and the bioelectrical impedance measurement section 503 are provided in the measurement device 10, and the input section 501, the memory section 504, the control section 505, and the output section 506 are provided in the user terminal 20.

The input section 501 receives operation input from the user. In this embodiment, in particular, the height, age, and gender of each user are input to the input section 501. The operation buttons 18A to 18D of the measurement device 10 and the touch panel of the user terminal 20 can all serve as the input section 501. The weight measurement section 502 corresponds to the load cell of the measurement device 10.

The bioelectrical impedance measurement section 503 has each of the electrodes 111R, 111L, 112R, 112L, 161R, 161L, 162R, and 162L provided by the measurement device 10, the energizing electrodes 161R, 161L, 111R, and 111L, and a current control circuit for applying a weak AC constant current to those electrodes. The current control circuit is capable of applying AC constant current at a plurality of different frequencies.

The memory section 504 stores, for each user, the information input from the input section 501, the weight measured by the weight measurement section 502, the bioelectrical impedance measured by the bioelectrical impedance measurement section 503, and the body composition calculated by the control section 505. The memory section 504 also stores the measurement program for measuring weight and body composition and the health level determination program of this embodiment, as well as data generated by those programs and various information (e.g., inference models to be described later) used for those programs.

The control section 505 controls each section of the health level determination system 510 in accordance with the program for measurement, and also calculates the body composition and determines the health level based on the information input to the input section 501, the weight measured by the weight measurement section 502, and the bioelectrical impedance measured by the bioelectrical impedance measurement section 503 in accordance with the health level determination program. The output section 506 corresponds to the display panel 17 of the measurement device 10 or the touch panel of the user terminal 20. The output section 506 displays, according to the control of the control section 505, a screen for inputting information to the input section 501, a screen for controlling the control section 505, a screen showing the results of calculations by the control section 505, and the like. The program for measurement and the program for determining the health level may be provided to the measurement device 10 or the user terminal 20 by being downloaded by the user terminal 20 via a communication network, or the program for measurement and the program for determining the health level may be recorded on a non-temporary recording medium and may be read by the measurement device 10 or the user terminal 20 from the recording medium.

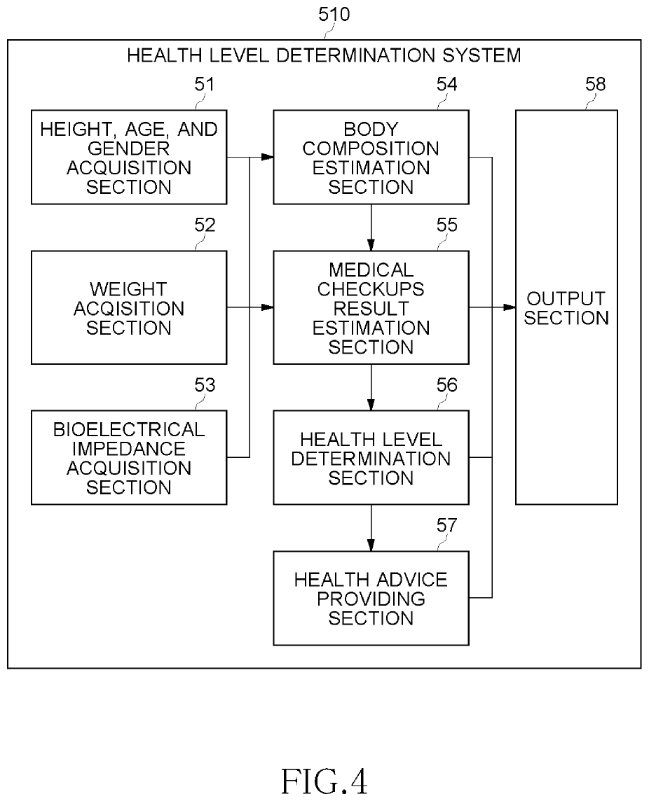

FIG. 4 shows a block diagram showing the functional configuration of the health level determination system according to the first embodiment of the present disclosure. In the health level determination system 510, various functions are realized by the control section 505 executing various programs. FIG. 4 shows, in particular, the functions that are realized by executing the health level determination program of this embodiment. The health level determination system 510 is equipped with a height, age, and gender acquisition section 51, a weight acquisition section 52, a bioelectrical impedance acquisition section 53, a body composition estimation section 54, medical checkup result estimation section 55, a health level determination section 56, a health advice providing section 57, and an output section 58.

The height, age, and gender acquisition section 51 acquires height, age, and gender as the user's biometric information by accepting the user's operation input at the input section 501. The weight acquisition section 52 acquires the weight as the user's biometric information by measuring the user's weight at the weight measurement section 502. The bioelectrical impedance acquisition section 53 measures the bioelectrical impedance of the user's whole body and each body part (hereinafter referred to as “body part” including the whole body).

The bioelectrical impedance acquisition section 53 measures, for example, the bioelectrical impedance of a plurality of body parts of the user in the following manner:

(1) To measure the bioelectrical impedance of the whole body, a current is supplied using the energizing electrode 161L and the energizing electrode 111L, and the potential difference between the measuring electrode 162L in contact with the left hand and the measuring electrode 112L in contact with the left foot is measured in the current path flowing through the left hand, the left arm, the chest, the abdomen, the left leg, and the left foot;
(2) To measure the bioelectrical impedance of the right leg, the current is supplied using the energizing electrode 161R and the energizing electrode 111R, and the potential difference between the measuring electrode 112L in contact with the left leg and the measuring electrode 112R in contact with the right leg is measured in the current path flowing through the right hand, the right arm, the chest, the abdomen, the right leg, and the right foot;
(3) To measure the bioelectrical impedance of the left leg, the current is supplied using the energizing electrode 161L and the energizing electrode 111L, and the potential difference between the measuring electrode 112L in contact with the left leg and the measuring electrode 112R in contact with the right leg is measured in the current path flowing through the left hand, the left arm, the chest, the abdomen, the left leg, and the left foot;
(4) To measure the bioelectrical impedance of the right arm, the current is supplied using the energizing electrode 161R and the energizing electrode 111R, and the potential difference between the measuring electrode 162L in contact with the left hand and the measuring electrode 162R in contact with the right hand is measured in the current path flowing through the right hand, the right arm, the chest, the abdomen, the right leg, and the right foot; and
(5) To measure the bioelectrical impedance of the left arm, the current is supplied using the energizing electrode 161L and the energizing electrode 111L, and the potential difference between the measuring electrode 162L in contact with the left hand and the measuring electrode 162R in contact with the right hand is measured in the current path flowing through the left hand, the left arm, the chest, the abdomen, the left leg, and the left foot.

In the case of a system that has no electrodes in contact with the hands but only electrodes in contact with the left and right feet, although only the bioelectrical impedance of one body part (bioelectrical impedance corresponding to the whole body, which is different in the measurement points from those of a system with electrodes in contact with the hands) is measured, it is possible to estimate the body composition of multiple items.

In this way, the bioelectrical impedance acquisition section 53 passes an AC constant current from each energizing electrode to a predetermined part of the user's body and measures the potential difference generated in this current path. The bioelectrical impedance of the plurality of body parts of the user is then calculated based on the respective values of such current and potential difference. The configuration for measuring the bioelectrical impedance can be the same as that of a general body composition analyzer. The bioelectrical impedance of each of the plurality of body parts is calculated for each of the following conditions: when AC constant current of a reference frequency (e.g., 50 kHz) is applied, when AC constant current of a high frequency (e.g., 250 kHz) is applied, and when AC constant current of a low frequency (e.g., 5 kHz) is applied.

The body composition estimation section 54 acquires the height, age, and gender obtained by the height, age, and gender acquisition section 51, the weight obtained by the weight acquisition section 52, and the bioelectrical impedance obtained by the bioelectrical impedance acquisition section 53, and estimates the body composition of the user by a calculation using such information. The body composition estimation section 54 estimates the body composition of the user by applying the biometric information and bioelectrical impedance to a predetermined regression equation and performing a calculation to determine body composition information including the fat percentage, fat mass, lean mass, muscle mass, visceral fat mass, visceral fat level, internal fat area, subcutaneous fat mass, basal metabolic rate, bone mass, body water content, BMI (Body Mass Index), intracellular (BMI), intracellular fluid volume, extracellular fluid volume, and so on. In addition, the value of an index indicating the quality of muscles may be acquired. The same configuration for calculating the body composition information as that of a general body composition analyzer can be used. The body composition estimation section 54 outputs the acquired body composition information to the medical checkup result estimation section 55 and the output section 58.

The medical checkup result estimation section 55 obtains the biometric information including the height, age, and gender obtained by the height, age, and gender obtaining section 51 and the weight obtained by the weight obtaining section 52, and the body composition information calculated by the body composition estimation section 54 and estimates the medical checkup result based on the information.

In medical checkups, various items are examined. For example, according to the “1-Day Complete Medical Checkup Basic Test Items 2018 Version” of the Japanese Society of Ningen Dock (human medical checkups), the essential items of medical checkups include physical measurement, physiological test, X-ray/ultrasound, blood test, urine test, stool test, interview for medical care, physical examination by physician(s), explanation of the results, and health guidance (see https://www.ningen-dock.jp/wp/wp-content/uploads/2018/06/1-Day-Complete-Medical-Checkup-Basic-Test-Items.pdf). The health checkup result estimation section 55 estimates the health checkup result from the body composition information, wherein as to the body measurements necessary for the estimation, height and weight, etc. are already obtained as biometric information.

In addition, it is known that blood tests, especially those items related to biochemistry which includes total protein, albumin, creatinine, estimated glomerular filtration rate (eGFR), uric acid, total cholesterol, HDL cholesterol, LDL cholesterol, Non-HDL cholesterol, triglyceride, total bilirubin, aspartate aminotransferase (AST), alanine aminotransferase (ALT), gamma-glutamyl transpeptidase (γ-GT), alkaline phosphatase (ALP), fasting blood sugar, hemoglobin A1c (HbA1c), have a relatively strong correlation with body composition. The health checkup result estimation section 55 in this embodiment, in estimating the health checkup results from the body composition, particularly estimates the values of some or all of the biochemical test items above that have a relatively strong correlation with the body composition among the health checkup items.

The medical checkup result estimation section 55 further estimates the items that correspond to hematology and serology among the essential items of the medical checkups. The hematology as a test item includes red blood cell count, white blood cell count, hemoglobin, hematocrit, MCV (Mean Corpuscular Volume), MCH (Mean Corpuscular Hemoglobin), MCHC (Mean Corpuscular Hemoglobin Concentration), and platelet, and serology as a test item includes CRP (C-Reactive Protein), blood typing (ABO/Rh), and HBsAg (Hepatitis B surface antigen). The medical checkup result estimation section 55 estimates some or all of these.

The following describes the relationship between biometric information and body composition information and biochemical test values before explaining operations of the medical checkup result estimation section 55.

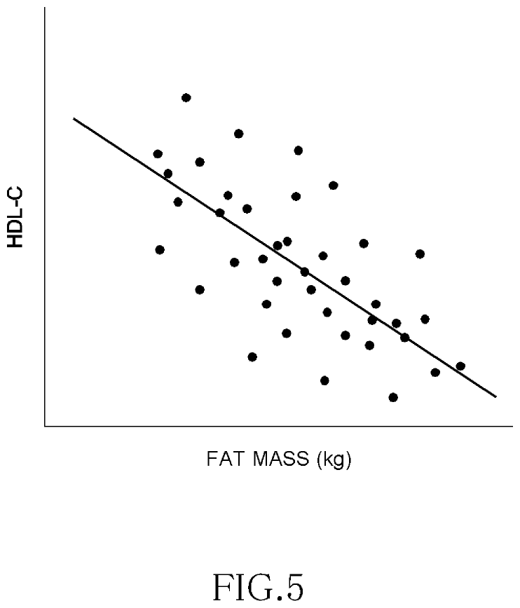

FIG. 5 is a graph showing the correlation between HDL cholesterol (vertical axis), which is a biochemical test value according to the first embodiment of the present disclosure, and fat mass (kg) (horizontal axis), which is body composition information. As can be seen from the graph of FIG. 5, there appears to be a linear correlation between the HDL cholesterol and the fat mass, where the larger the fat mass, the smaller the HDL cholesterol, but the variation is relatively large, and it seems difficult to estimate the HDL cholesterol from the fat mass with high accuracy.

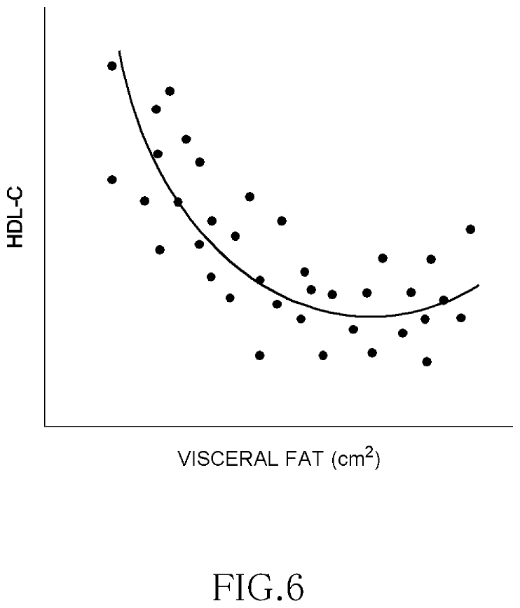

FIG. 6 is a graph showing the correlation between HDL cholesterol (vertical axis) and amount of visceral fat (cm2) (horizontal axis), which is body composition information, in the first embodiment of the present disclosure. As can be seen from the graph in FIG. 6, there appears to be a correlation between HDL cholesterol and the square of the amount of visceral fat, but even in this case, the correlation is weak and the variation is too large to estimate HDL cholesterol from the amount of visceral fat.

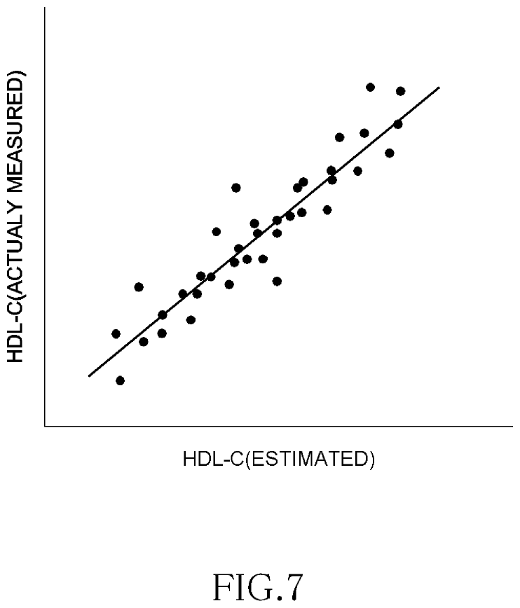

FIG. 7 is a graph showing the accuracy of the estimation using the multi-item body composition information of the first embodiment of the present disclosure. The vertical axis of the graph in FIG. 7 is the actually measured HDL cholesterol, and the horizontal axis is the estimated HLD cholesterol estimated by applying multiple regression analysis to the multi-item body composition information. As shown in FIG. 7, HDL cholesterol can be estimated accurately by performing statistical analysis such as multiple regression analysis using the body composition information of multiple items.

The medical checkup result estimation section 55 estimates the medical checkup results from the biometric information and body composition information using a multiple regression equation obtained by performing a statistical analysis such as a multiple regression analysis on a sufficient number of data set of biometric information, body composition information and the medical checkup result. This multiple regression equation uses the biometric information and body composition information as explanatory variables and the medical checkup results as the objective variables. The multiple regression equation can also be called an inferential model (or inference model) obtained by learning from a large number of data set of biometric, body composition information and medical checkup results obtained from actual medical checkups as training data. The inference model is not limited to a multiple regression equation, but can be any other inference model generated by learning, such as a decision tree or neural network. For example, for age and gender, different multiple regression equations can be prepared and used for each age and gender without using them as explanatory variables.

The medical checkup result estimation section 55, for example, estimates HDL cholesterol Y by multiple regression analysis using the following multiple regression equation (1) where the fat mass (kg) is x₁ and the visceral fat mass (cm²) is x₂. In order to increase the accuracy of the estimation of the biochemical test values, for example, HDL cholesterol Y may be estimated using a multiple regression equation in which the x₁ and x₂ are also added as explanatory variables with considering the amount of change and/or the direction of change of x₁ and x₂.

Y=ax₁/x₂²+b (1)

The medical checkup result estimation section 55 stores a plurality of multiple regression equations as described above for estimating biochemical test values of multiple items. Sets of biometric information and body composition information used to estimate each biochemical test value are generally different for each biochemical test value to be estimated. The medical checkup result estimation section 55 estimates each biochemical test value by substituting the biometric information and body composition information required for this regression equation into the regression equation for estimating each biochemical test value. The medical checkup result estimation section 55 outputs the estimated medical checkup results to the health level determination section 56 and to the output section 58.

The health level determination section 56 determines the health level based on the estimated values of multiple items of medical checkup results estimated by the medical checkup result estimation section 55. However, as described above, in this embodiment, not all the results of all items obtained in a medical checkup actually conducted as medical checkup results are estimated, but especially biochemical test values are estimated. Therefore, the health level determination section 56 will determine the health level based on this information.

The health level determination section 56 may determine, as the health level, the overall disease risk of lifestyle-related diseases (hereinafter simply referred to as “disease risk”), which indicates the overall risk of lifestyle-related diseases or diseases related to the lifestyle-related diseases.

According to the conventional method, the disease risk is obtained by making a total score of, for example, chest X-ray, urine protein, urine occult blood, AST (GOT), γ-GT (γ-GTP), LDL cholesterol, HDL cholesterol, triglyceride, uric acid, HbA1c, FPG, hematocrit, red blood cell count, CRP, maximum blood pressure, minimum blood pressure, etc. are obtained by making a comprehensive score. The medical checkup result estimation section 55 of the present embodiment obtains the disease risk based, for example, on AST (GOT), γ-GT (γ-GTP), LDL cholesterol, HDL cholesterol, triglyceride, uric acid, HbA1c, FPG, hematocrit, and CRP, which are obtained by estimation in the medical checkup result estimation section 55.

The health level determination section 56 may obtain the disease risk as the health level following the conventional method of total scoring and by using a formula for calculating the overall score with modifications in corresponding to medical checkup results (e.g., chest X-ray, blood pressure, etc.) for which estimation results cannot be obtained by the medical checkup result estimation section 55. The modification may be a modification that simply omitting items for which the estimation results are not obtained by the conventional method of total scoring.

Here, the medical checkup results estimated from biometric and body composition information are less accurate than the medical checkup results obtained by actually conducting medical checkups, depending on the items. Therefore, if the disease risk is calculated from the estimated health checkup results using the total score calculation formula following the conventional method, the disease risk may deviate from the actual disease risk.

In order to make such cases less likely to occur, there is a method in which the health level determination section 56 determines the health level based on the medical checkup results estimated by the medical checkup result estimation section 55 using a multiple regression equation obtained by performing a statistical analysis such as multiple regression analysis on the data set of health levels and medical checkup results estimated from many sets of biometric information and body composition information. This multiple regression equation uses the medical checkup results estimated from the biometric information and body composition information as explanatory variables and the health level as the objective variable. This multiple regression equation can also be called an inferential model (or inference model) obtained by learning the pairs of medical checkup results estimated from a large number of sets of biometric and body composition information and the health level of the subject for whom the biometric and body composition information was obtained as training data. The inference model is not limited to a multiple regression equation, but can be any other inference model generated by learning, such as a decision tree or neural network. Also, for example, for age and gender, different multiple regression equations may be prepared and used for each age and gender without using them as explanatory variables. In the health level determination system 510, not only the estimated medical checkup results but also biometric information and body composition information are obtained, so that not only the estimated medical checkup results but also biometric information and body composition information may be used as explanatory variables.

As the health level among the training data to be used in this learning process, (1) the value of disease risk calculated by the conventional method for total scoring from the medical checkup results (including information such as chest X-ray) calculated from the results obtained by actually conducting medical checkups may be used, or (2) information on whether or not the person actually has a lifestyle-related disease may be used, or (3) information on actual medical expenses may be used, considering the medical expenses incurred as the presence or severity of a lifestyle-related disease. The user does not need to know where his or her health level is, for example, among the 10 levels. Even if the user only knows, for example, whether he or she is in the “normal range” or in the “dangerous range” with the high accuracy of determination, the user can recognize the risk of his or her health by the measuring instruments used in the home on a daily basis and the usefulness can be fully felt.

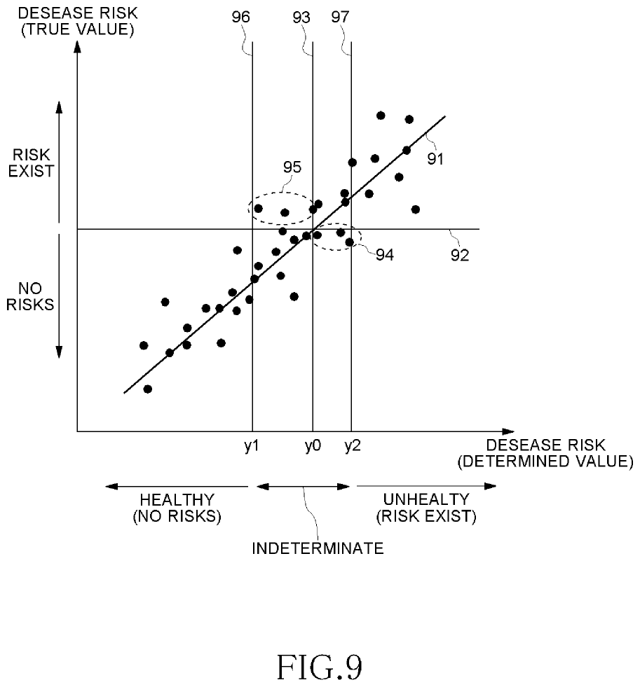

In any of (1) to (3), the multiple regression equation that outputs the degree of health by inputting the estimated medical checkup results is a different equation from the calculation equation used in the conventional method for total scoring. The multiple regression equation is adjusted by the above-mentioned learning to calculate an index that combine the items of the health checkup results based on the nature of the bioelectrical impedance to be measured using a different method from the conventional one so that the relationship between the estimated medical checkup results and the degree becomes stronger. The fact that the equation for determining the health level can be adjusted by using medical checkup results in this way means that even if the estimation accuracy of each of the multiple items in the medical checkup results is low based on the body composition information, the index of the health level that combines the multiple items can be adjusted from among a large number of combinations to ensure accuracy in limited situations (actual health risk from a specific perspective), which means that the determination of the user's health level by measuring the bioelectrical impedance can be realized more reliably. This will be explained again in detail referring FIG. 9.

The health level determination section 56 outputs the determined health level to the health advice providing section 57 and to the output section 58.

The health advice providing section 57 generates information for presenting the health level determined by the health level determination section 56 or information based on the health level to the user, and outputs the information to the output section 58.

The output section 58 displays the body composition obtained by the body composition estimation section 54, the medical checkup results estimated by the medical checkup result estimation section 55, the health level determined by the health level determining section 56, and the information generated by the health advice providing section 57. Each health information may be switched and displayed respectively, or may be displayed collectively.

As described above, the storage section 504 and the control section 505 may be provided in either the measurement device 10 or the user terminal 20, but one or both of the storage sections 504 and the control sections 505 may be provided in a server that can communicate with the user terminal 20 via a communication network. In the case where the measurement device 10 has a function to communicate with the communication network, the storage section 504 and the control section 505 may be provided in the server, thereby eliminating the need for the user terminal 20. That is, the body composition estimation section 54, the medical checkup result estimation section 55, the health level determining section 56, and the health advice providing section 57 may be provided in a server that can communicate with the measurement device 10 or the user terminal 20 via a communication network.

### Second Embodiment

The health level determination system of the second embodiment estimates a plurality of body compositions from bioelectrical impedances of a plurality of body parts, and determines a health level based at least partially on the estimated plurality of body compositions.

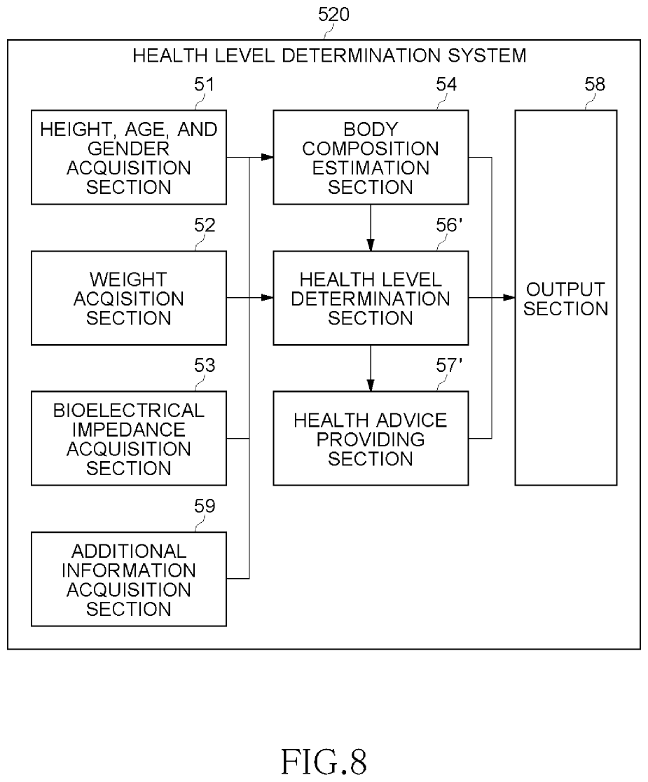

FIG. 8 is a block diagram showing a functional configuration of a health level determination system according to the second embodiment of the present disclosure. In the health level determination system 520, the same number is assigned to the same configuration as that of the health level determination system 510 of the first embodiment, and explanations are omitted as appropriate. The health level determination system 520 has a height, age, and gender acquisition section 51, a weight acquisition section 52, a bioelectrical impedance acquisition section 53, a body composition estimation section 54, a health level determination section 56′, a health advice providing section 57′, an output section 58, and an additional information acquisition section 59.

The configuration and operation of the height, age, and gender acquisition section 51, the weight acquisition section 52, the bioelectrical impedance acquisition section 53, and the body composition estimation section 54 are the same as those in the first embodiment. The body composition estimation section 54 outputs the acquired body composition information to the health level determination section 56′ and the output section 58.

The health level determination section 56′ determines the health level based on the information of height, age, and gender acquired by the height, age, and gender acquisition section 51, the information of weight acquired by the weight acquisition section 52, and the body composition information acquired by the body composition estimation section 54. The health level determination section 56′ may determine a disease risk as the health level, as in the first embodiment.

As described above, according to the conventional methods, the disease risk may be determined by making a total score of, for example, chest X-ray, urine protein, urine occult blood, AST (GOT), γ-GT (y-GTP), LDL cholesterol, HDL cholesterol, triglycerides, uric acid, HbA1c, FPG HbA1c, FPG, hematocrit, red blood cell count, CRP, maximum blood pressure, minimum blood pressure, etc. However, the health level determination section 56′ in the second embodiment does not estimate any of these medical checkup results, but determines the disease risk based on biometric information and body composition.

The health level determination section 56′ determines the health level from the biometric information and the body composition information using a multiple regression equation obtained by performing a statistical analysis, such as multiple regression analysis, on a large number of data set of biometric information, body composition information, and the health level. This multiple regression equation uses biometric information and body composition information as explanatory variables, and the health level as the objective variable. This multiple regression equation can also be called an inferential model (or inference model) obtained by learning from data set of biometric information, body composition information, and the health level calculated from the results obtained by actually conducting medical checkups on subjects who have obtained the biometric information and body composition information, using the data set as training data. As in the first embodiment, the inference model is not limited to a multiple regression equation, but can be any other inference model generated by learning, such as a decision tree or neural network. Also, for example, for age and gender, different multiple regression equations may be prepared and used for each age and gender without using them as explanatory variables.

FIG. 9 is a graph showing the accuracy of the determination of the health level using the multi-item body composition information of the second embodiment of the present disclosure. The vertical axis of the graph in FIG. 9 is, for example, a total score (true value) of a disease risk calculated by a conventional method from medical checkup results, and the horizontal axis is a score (determined value) of a disease risk obtained by inputting the multi-item body composition information into a multiple regression equation by the health level determination section 56′. In this graph, the line 91 represents a function showing the relationship between the true value and the corresponding determined value, and the closer a point on the graph is to the line 91, the closer the determined value to the true value is obtained by the determination (the higher the determination accuracy).

As the health level (true value) on the vertical axis in FIG. 9, (1) the value of disease risk calculated by the conventional method for total scoring based on the actual medical checkup results including information such as chest X-ray, (2) information on whether or not the person has a lifestyle-related disease may be used, or (3) information on actual medical costs may be used, considering the medical costs incurred as the presence or severity of lifestyle-related diseases. The health level (determined value) on the horizontal axis of FIG. 9 may be a value obtained by inputting multiple items of body composition information into a multiple regression equation, or a value obtained by inputting one or more items of body composition information and one or more items of biometric information into a multiple regression equation.

Here, if the boundary 92 between healthy and unhealthy is set at the true value of disease risk (health level), the boundary 93 of the determined value corresponding to the boundary 92 is obtained using the line 91. The determined value at this boundary 93 is y0. In the example in FIG. 9, there is a case where the determined value is greater than y0 but the true value is determined to be healthy, i.e., a false positive 94 shown in FIG. 9. In addition, there is a case where the determined value is less than y0 but the true value is determined to be unhealthy, i.e., false negative 95 shown in FIG. 9.

Therefore, the health level determination section 56′ sets the boundary 96 corresponding to the determined value y1 to include the false negative 95, and sets the boundary 97 corresponding to the determined value y2 to include the false positive 94, with the boundary 93 as the center, and sets the range from y2 to y1 as the indeterminate region. The indeterminate region does not necessarily need to include all false positives and all false negatives in the training data, but it should be set so as to reduce false positives and false negatives as much as possible.

In this way, the health level determination section 56′ of the present embodiment determines the health level in three values (three categories) of healthy, unhealthy, and indeterminate, but it is desirable that the area set as described above and corresponding to the determination of indeterminate be made as narrow as possible. For this purpose, the multiple regression equation in which the values of multiple items of body composition information (and biometric information) are input is adjusted in the direction in which the indeterminate region is narrowed by conducting statistical analysis including learning as described above. This narrowing of the indeterminate region means that the accuracy of discriminating between “health risk exists (dangerous range)” and “no health risk exists (normal range)” can be increased, making it possible to use a body composition analyzer for daily use to determine a useful health level.

The determination of the health level in three categories and the adjustment of the multiple regression equation for determining the health level so that the indeterminate region becomes narrow (and even more, the health level can be determined in two categories: healthy and unhealthy) are similarly applied to the health level determination section 56 in the first embodiment. By applying the method described below to the health level determination section 56 in the first embodiment (using the medical checkup results estimated from the body composition information) and to the health level determination section 56′ in the second embodiment (directly using the body composition information), the indeterminate region can be further narrowed.

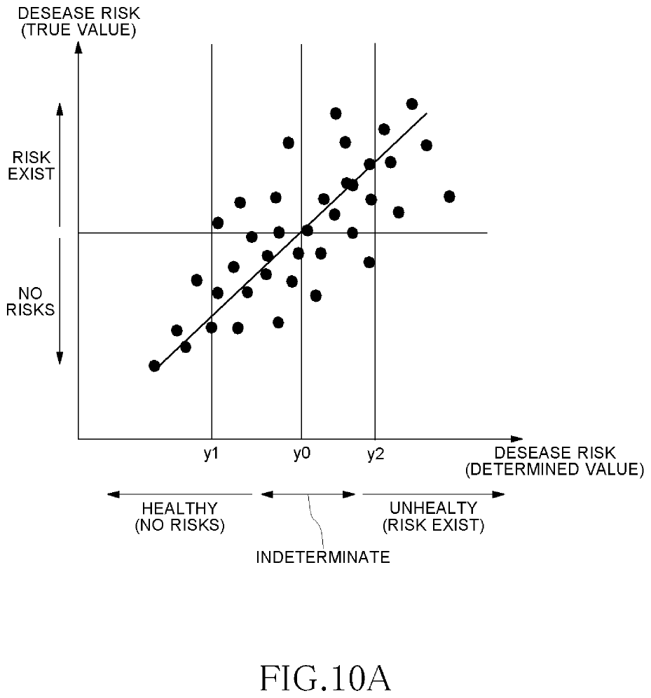

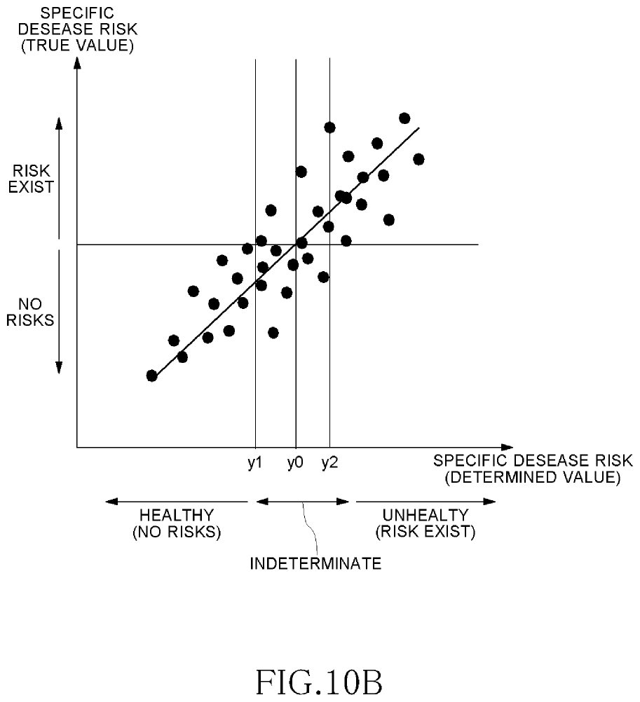

FIGS. 10A and 10B are graphs showing an example (of the second embodiment) in which the health level (true value) is the vertical axis and the health level (determined value) calculated directly based on body composition information and biometric information such as body fat percentage, visceral fat, muscle mass, basic metabolism, age, and gender is the horizontal axis. This method not only adjusts the multiple regression equation for calculating the health level (determined value) based on the medical checkup results estimated from the body composition information or the body composition information itself (which is the horizontal axis in FIG. 9), but also adjusts the equation for calculating the health level (true value), which is the basis for adjusting the multiple regression equation (which is the vertical axis in FIG. 9). In other words, while changing not only the equation for calculating the health level (determined value) on the horizontal axis but also the equation for calculating the health level (true value) on the vertical axis, we search for a state in which the many sets of data to be plotted are as close as possible to the line 91 (the number of data points that become false positives 94 and false negatives 95 and the distance from the line 91 are reduced).

FIG. 10A shows a graph in which the disease risk assessment value obtained by the conventional total score calculation formula from the actual medical checkup results is directly adopted as the true value, and FIG. 10B shows a graph in which the disease risk assessment value obtained by the above search is adopted as the true value.

For this search, if the formula for determining the health level (true value) on the vertical axis is changed on a trial basis, the plot on the vertical axis will be made with a different value as the true value from the value obtained by the conventional formula for calculating the total score from the actual medical checkup results. What value is taken as the true value, i.e., what formula is used to obtain the health level (true value), is determined so as to have strong correlation to the information on whether or not the examinee with the body composition information (and biometric information) actually has a lifestyle-related disease, and/or the medical expenses actually incurred by the examinee with the body composition information (and biometric information) (disease type). The medical expense may be extracted by limiting the type of disease or the overall medical expense. Such a health level (true value) evaluates the risk of related diseases by a combination of indices that have a high affinity with body composition, such as TG, LDL-C, HDL-C, HbA1c, etc.

As shown in FIG. 10A, if the health level (true value) is determined without adjusting the vertical axis based on the risk determination from the general health checkup results, the error margin would be large even when there is a relationship between the vertical and horizontal axes, because such health level (true value) includes indicators that do not have a strong correlation with the body composition information that is the basis of the health level (determined value) on the horizontal axis. On the other hand, in order to obtain the degree of health (true value) in accordance with whether or not a particular disease is actually being suffered and whether or not medical expenses are actually being spent, the method of determining the degree of health (true value) is adjusted from a combination of indicators with high affinity to body composition information so that the error is minimized to the maximum extent possible, thereby as shown in FIG. 10B, it is possible to increase the effectiveness of determining the health level from body composition information.

In the example shown in FIG. 10B, the result in which a high degree of correlation was obtained by limiting the disease risk to be the health level, as a result of the search. Thus, if selecting a disease that is strongly related to body composition (e.g., arteriosclerosis) as the specific disease to be focused on in adjusting the method of determining the health level (true value), the effectiveness of the determination is effectively increased. In other words, the health level determination section 56 or 56′ determines, for example, the metabolic caution risk indicating the risk of metabolic syndrome, the vascular system damage risk indicating the risk of damage to blood vessels, the abnormal glucose metabolism (diabetes) risk indicating the risk of abnormal glucose metabolism (diabetes), and the hepatic dysfunction or liver disease risk, the risk of sarcopenia and frailty, which indicates the risk of muscle weakness and physical frailty, in addition to the disease risk, it would be highly effective.

When these health levels are determined by conventional methods using a physician's judgment or a predetermined algorithm, the risk of metabolic syndrome can be generally determined from blood lipids (TG, TC, LDL-C, HDL-C), glucose tolerance index (fasting blood glucose, HbAic, insulin), blood pressure, and chest measurement; vascular damage risk can be determined from blood lipids, blood pressure, glucose tolerance, and lifestyle habits such as smoking habits; glucose metabolism (diabetes) risk can be determined from HbA1c, blood glucose (fasting), and body mass index; liver function risk can be determined from AST (GOT), ALT (GPT), γ-GTP; and sarcopenia and frailty risk can be determined from total body muscle mass, body weight, grip strength, height, walking speed, gait, center of gravity sway, and lower limb muscle strength.

In the case of determining these health levels by the first embodiment, the various information described above, which is the basis for determining the health level, is estimated from the body composition information (and biometric information). For example, blood lipids, blood pressure, abdominal circumference, some lifestyle factors, grip strength, walking speed, gait, and center of gravity sway can be estimated relatively accurately using body composition information (and biometric information), and obesity, total body muscle mass, body weight, height, and lower limb muscle strength can be obtained as body composition information (and biometric information) itself.

In contrast, by the second embodiment, these health levels must be determined directly from the body composition information (and biometric information), but in some cases, the second embodiment may be able to make determinations with a narrower indeterminate region than the first embodiment. In particular, since the risk for diseases whose probability of incidence is affected by the way we lead our daily lives can be measured naturally on a daily basis under normal conditions, it can be expected that direct determination from body composition, which reflects minute daily changes, is more suitable for detecting the original warning signals. Therefore, for example, even if it is difficult to accurately estimate each of the liver function indices required by a blood test in medical checkups from the body composition information (and biometric information), the health level determination system 520 can issue a warning signal indicating the liver function caution risk “exists” by capturing the state and time variation of the body composition that is measured on a daily basis.

Whether or not a useful determination can be made for the user depends on the inference model described above. Therefore, it is advantageous to select an appropriate type of inference model (multiple regression analysis, principal component analysis, decision tree, neural network, Bayesian network, etc.) and to prepare a good and sufficient amount of training data and perform proper learnings. As good and sufficient supervisory data, data sets of body composition information (and biometric information) and the health level (true value) obtained from the information at the time when the body composition was measured (the total score based on the results of the medical checkups actually performed at that time, the presence or absence of an actual disease at that time, or the actual medical expenses at that time, etc.) should be prepared. In addition to the above, by preparing data sets of the body composition information (and biometric information) measured at many points in a given period and the health level (true value) obtained from the information at the end of the period, it is possible to issue a warning signal indicating that health risk “exists” with high accuracy based on the daily changes in the body composition measured on a daily basis.

Furthermore, as explained referring FIGS. 10A and 10B, the usefulness of the system can be improved by adjusting the health level (true value) used as the training data so that it is highly correlated with the body composition information. For example, in the case of the vascular damage risk, instead of directly using the value obtained by conventional methods from information on blood lipids, blood pressure, glucose tolerance, smoking habits, or the like obtained from an actual medical checkups as the true value, an inference model can be obtained by adjusting method of determining the true value in accordance with whether or not the symptoms of arteriosclerosis are actually advanced, whether or not medical expenses for treating arteriosclerosis are actually spent, or the like so that the degree of coincidence between the true value and the value determined from body composition information (and biometric information) becomes high.

Some of the health level, for example, the risk of sarcopenia and frailty, which is determined by a ratio of total body muscle mass to body weight and a ratio of total body muscle mass to height, can be determined by a theoretically defined function or algorithm (rule-based) without using an inference model. Such a health level can be determined by directly using biometric data and body composition data. In other words, it is possible to use the muscle mass in the body composition information as the total body muscle mass to determine the risk of sarcopenia and frailty.

If the body composition estimation section 54 has a function for estimating an evaluation of the density of muscle fibers, the fullness of muscles, and the cell size of muscles based on the frequency characteristics of electrical resistance, the ratio of resistance and reactance, the phase angle, and the like, this evaluation value may be used for determining the sarcopenia and frailty risk. In the body composition analyzer commercialized by the applicant, this evaluation value is output as the number of muscle quality points (trademark). Thus, even when not using inferential models (inference models), determinations based on body composition information may be more direct and accurate than determinations based on medical checkup results.

Of course, the risk of sarcopenia and frailty can also be determined by an inference model. In this case, instead of assuming the value obtained by the rule-based method to be the health level (true value), the true value is set in accordance with whether or not the symptoms of sarcopenia have actually progressed, whether or not medical expenses to treat sarcopenia have actually been spent, etc., and the inference model is calculated so that the degree of coincidence with the value determined from body composition information (and biometric information) is high. Since the body composition information used for the inference model includes not only indices that indicate the state of muscles but also indices that indicate the state of fat, it is possible to determine the risk of sarcopenia and frailty with higher accuracy.

As described above, the health level determination section 56 or 56′ of the embodiment determines each of the above health levels in three values including risk exists, no risk exist, and indeterminate, or even in two values including risk exists and no risk exist if the indeterminate region can be sufficiently narrowed. If the health level is obtained as a continuous value, the determination can be made by dividing the range of such continuous values into the order of healthy (no risk), indeterminate, and unhealthy (risk exist). When a probabilistic model such as a Bayesian network is used as an inference model, it is not necessarily required to obtain the health level as a continuous value, but health (no risk), indeterminate, and unhealthy (risk exist) can be determined according to the obtained probability.

The health level determination section 56 or 56′ outputs the determined health level to the health advice providing section 57 or 57′ and the output section 58.

The health advice providing section 57 or 57′ determines health advice according to the health level determined by the health level determination section 56 or 56′. The health advice is output to the output section 58 as screen information and is output (displayed) on the output section 58. The health advice providing section 57 or 57′ generates the screen information to notify the user of the result of the determination of the health level if the result is healthy or unhealthy, while generates the screen information to request additional information about the user if the result of the determination is indeterminate.

As additional information, the user can easily add information by requesting lifestyle habits (drinking habits, smoking habits, sleeping hours, etc.) as well as information that can be measured with measurement devices at home without going to a medical facility, such as numerical values of activity meters, blood pressure meters, and sleep meters. A screen for inputting additional information is generated by the health advice providing section 57 or 57′ and displayed by the output section 58, thereby constituting the additional information acquisition section 59. The user can input additional information via the additional information acquisition section 59. The additional information acquisition section 59 may request heartbeat-related information such as heart rate (pulse rate) heartbeat fluctuation, or the like as additional information. The information related to the heartbeat may be obtained using a wearable device equipped with a heartbeat sensor or the like, or the measurement device 10 of the health level determination system 510 may calculate the heartbeat rate from the bioelectrical impedance.

The health level determination section 56 or 56′ determines the health level using the additional information added to the additional information acquisition section 59. In this case, if the health level based on the body composition information is obtained as a continuous value, the health level (continuous value) may be adjusted by adding a modification based on the additional information to the health level. In the case where the health level based on the body composition information is obtained by a probability model, an inference model in which the biometric information, the body composition information, and the additional information are explanatory variables (inputs) and the health level is an objective variable (output) can be used for the health level determination section 56 or 56′. The health level determination section 56 or 56′ may determine the health level in the three value of healthy, unhealthy, or indeterminate even when additional information is given, or it may determine the health level in the two value of healthy or unhealthy without determining as indeterminate when additional information is given. Alternatively, the health level determination section 56 or 56′ may make a determination with three values: healthy, need attention, and unhealthy.

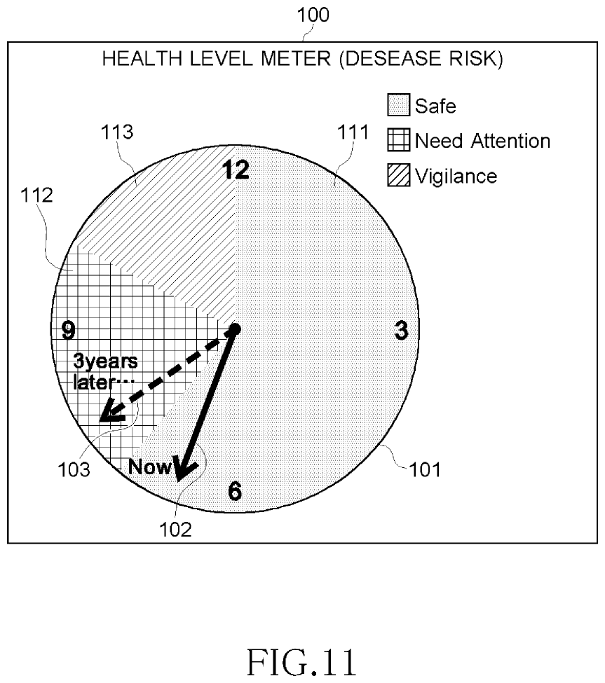

FIG. 11 shows an example of a health advice screen in accordance with an embodiment of the present disclosure. The output section 58 displays the health advice screen. The health advice providing section 57 or 57′ generates screen information for displaying the health advice screen on the output section 58. In the example of FIG. 10, the health level determination section 56 or 56′ determines the health level in three values: healthy, need attention, and unhealthy, and the determined health level is shown as the advice “Safe”, “Need Attention”, and “Vigilance”, respectively, in the health advice screen 100.

FIG. 11 shows the result of determining the disease risk as a health level. In the health advice screen 100, the disease risk is indicated by a clock-like dial 101 and hand 102, which are called health level meters. In the dial 101, the fan-shaped area from 0:00 to a predetermined time is designated as the safe area 111, the fan-shaped area following the safe area 111 to a predetermined time is designated as the need attention area 112, and the fan-shaped area following the need attention area 112 to 12:00 (or 0:00) is designated as the alert area 113. The hand 102 shows the determination result. In the example of FIG. 10, the determination result is “safe”, which is relatively close to “need attention”.

In the health advice screen 100, the predicted value three years from now is also indicated by the hand 103. The predicted value three years from now may be obtained by advancing a predetermined time (e.g., 1.5 hours) from the current determined value (hand 102) in the health advice screen 100, taking into account the age, gender, etc., or, if there is a history of determined values in the past, the predicted value may be obtained based on such history. The health level meter may also indicate how far the hand will return in the health meter by taking an improvement action.

The health advice providing section 57 or 57′ generates the health advice screen 100 of the health level meter in the same manner according to the results of the determination in the health level determination section 56 or 56′ for other health levels, and the output section 58 displays it.

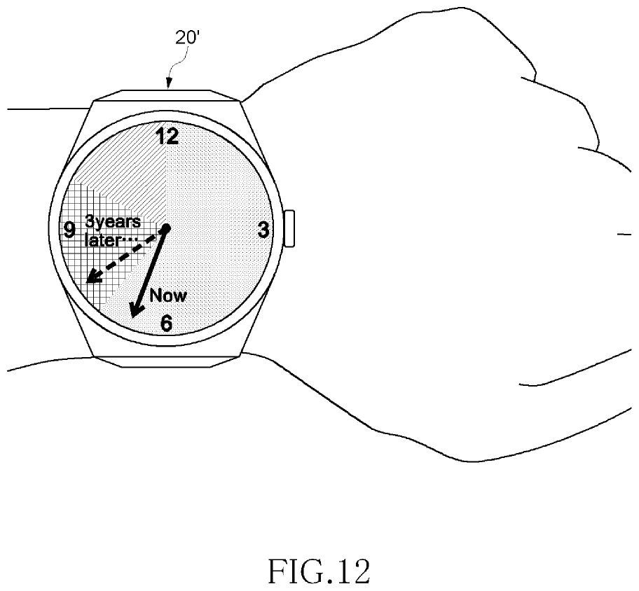

FIG. 12 shows another example of a health advice screen in accordance with the embodiment of the present disclosure. In FIG. 12, an example is shown in which the user terminal 20 is a wearable information processing device. Specifically, the user terminal 20′ as a wearable information processing device is a smart watch or smart wristband worn on the arm. In this example, the user terminal 20′ is equipped with a liquid crystal display device in the dial part, and this display device corresponds to the output section 506 or output section 58. In this case, for example, among the configurations shown in FIG. 3, the input section 501, the weight measurement section 502, the bioelectrical impedance measurement section 503, the memory section 504, and the control section 505 are provided in the measurement device 10, and the output section 506 is provided in the user terminal 20′.

The user terminal 20′ is capable of communicating with the measurement device 10 by Near Field Communication (NFC), and screen information indicating the health level meter is transmitted from the measurement device 10 to the user terminal 20′. The user terminal 20′ can switch between the clock display and other information display and the display of the health level meter.

The user terminal 20′ may be equipped with an accelerometer for measuring the acceleration of the user's body. The user terminal 20′ may also be equipped with a pulse meter for detecting the pulse of the user. The user terminal 20′ may move the hand of the health meter by combining information such as the user's body movement and activity tendency detected by the accelerometer, the energy consumption, and the pulse rate measured by the pulse meter. For example, if the user exercises a lot and the energy consumption is high, the hand may move back to the safe direction. Conversely, if the user exercises less than the average, the hand may move forward in the risky direction.

The user terminal 20′ as a wearable information processing device may be used as a second user terminal, paired with a handheld user terminal 20 as a first user terminal. In this case, for example, the weight measuring section 502 and the bioelectrical impedance measuring section 503 are provided in the measurement device 10, the input section 501, the memory section 504, and the control section 505 are provided in the user terminal 20, and the output section 506 is provided in the user terminal 20 and the user terminal 20′. The terminal that displays the health level meter can then be switched between the user terminal 20 and the user terminal 20′.

As described above, according to the health level determination system 520 of the second embodiment, the health level can be determined without estimating the medical checkup results from the body composition information. Therefore, errors in the estimation of the medical checkup results from the body composition information and errors in the determination of the health level from the medical checkup results are not superimposed, and the accuracy of the health level determination can be improved.

In addition, since the health level determination system 510 or 520 of the embodiment determines the health level in three values: healthy, indeterminate, and unhealthy, it is possible to make a definitive determination when there is no (or small) suspicion of a false positive or false negative, and to determine it as indeterminate when there is a suspicion of a false positive or false negative. In addition, since the health level determination system 50 of the embodiment requests additional information when the determination result is indeterminate and does not request additional information when the determination can be made definitively without additional information, the health level can be determined simply. Furthermore, even if the result of the determination is indeterminate, by requesting information that can be obtained at home, such as the numerical value of the activity meter or the numerical value of the blood pressure monitor, as additional information, the result of the determination of the health level can be obtained without going to a medical facility.

In addition, since the health level determination system 510 or 520 of the embodiment displays the results of the health level determination on a health advice screen 100 that includes a clock-like graphic, the user can intuitively grasp his/her own health level by checking the health advice screen 100. As described above, the health advice screen 100 may be displayed on the user terminal 20, on the user terminal 20′, or on the display panel 17 of the measurement device 10 (in this case, the user can know his/her own health level just by riding on the measurement device 10).

### Third Embodiment

The health level determination system of the third embodiment determines the health level based at least partially on the bioelectrical impedance of a plurality of body parts. In the second embodiment, the health level was determined from the body composition information, which is calculated by estimation based on the user's biometric information (height, weight, etc.) and the bioelectrical impedance of multiple body parts. Therefore, by determining the health level from the primary information, the biometric information and the bioelectrical impedance of multiple body parts, it may be possible to determine the health level more accurately than the second embodiment.

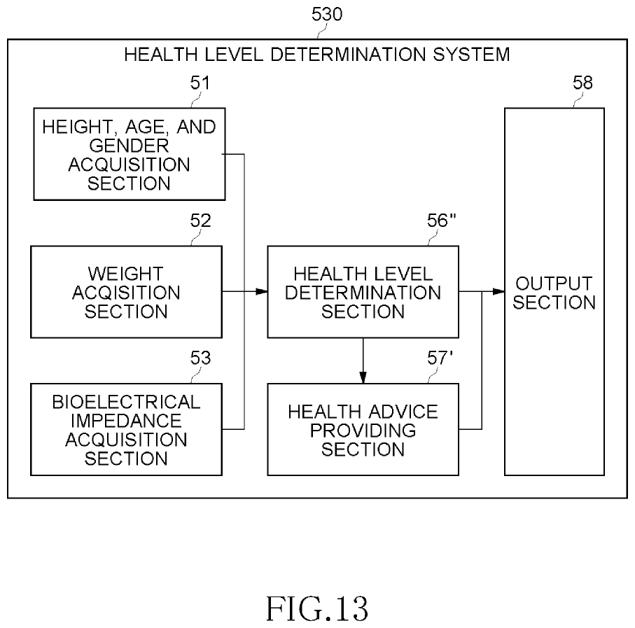

FIG. 13 is a block diagram showing a functional configuration of the health level determination system 530 of the third embodiment of the present disclosure. In the health level determination system 530, the same number is assigned to the same configuration as that of the health level determination system 520 of the second embodiment, and the description is omitted as appropriate. The health level determination system 530 is provided with a height, age, and gender acquisition section 51, a weight acquisition section 52, a bioelectrical impedance acquisition section 53, a health level determination section 56″, a health advice providing section 57″, and an output section 58.

As described above, in the bioelectrical impedance acquisition section 53, bioelectrical impedances of five body parts, namely, the whole body, right leg, left leg, right arm, and left arm, are obtained for high-frequency and low-frequency currents, respectively, and ten kinds of impedance values are conveniently obtained.

The health level determination section 56″ uses some or all of these 10 impedance values and biometric information (height, weight, etc.) to determine the health level. The health level determination section 56″ uses an inference model to determine the health level. In this inference model, biometric information and impedance values are used as explanatory variables (inputs), and the health level is the objective variable (output). To learn this inference model, a large number of data set of biometric information, impedance values, and health level (presence/absence of disease, medical expenses, etc.) can be used as training data.

The health level determination section 56″ of the third embodiment determines the health level in three values: healthy, unhealthy, and indeterminate, as in the second embodiment. As in the second embodiment of the health level determination system 520, the health level determination system 530 may request additional information when the result of the determination is indeterminate, and make a more accurate determination based on the additional information. Also, as in the second embodiment, a body composition estimation section 54 may be provided to estimate the body composition based on the biometric information and bioelectrical impedance.

All that has been described about the relationship between body composition information (and biometric information) and various health levels (true values, determined values) in the second embodiment can be applied to the relationship between bioelectrical impedance (and biometric information) and various health levels (true values, determined values) in the third embodiment. Everything explained above about the display of health levels and the configuration of the health advice screen can also be applied to the third embodiment.

As described above, the health level determination systems of the first through third embodiments determine the health level based at least partially, indirectly or directly, on bioelectrical impedance, so that the health level can be easily determined using a device such as a body composition analyzer.

In the above embodiments, an eight-electrode body composition analyzer was used as a body composition analyzer, but a four-electrode body composition analyzer for only feet or only hands may be used, or a body composition analyzer with nine or more electrodes may be used. In addition, although the accuracy is lower than that of a body composition analyzer, a simple body fat analyzer of the type in which a pair of current electrodes and voltage electrodes are held between the fingers or palms of both hands, or a body fat analyzer or subcutaneous fat analyzer of the type that measures the bioelectrical impedance of a limited body part such as a part of the upper arm or a part of the abdomen may be used.

### DESCRIPTION OF THE REFERENCE NUMERALS

10 Measurement device
11 Main unit
12 Handle unit
13 Connection cord
14 Housing section
15 Handle body
16L, 16R Grip
17 Display panel
18A to 18D Operation buttons
20, 20′ User terminal
50 Health information provision system
51 Height, age, and gender acquisition section
52 Weight acquisition section
53 Bioelectrical impedance acquisition section
54 Body composition estimation section
55 Medical checkup result estimation section
56, 56′, 56″ Health level determination section
57, 57′ Health advice providing section
58 Output section
59 Additional information acquisition section
91 line
92, 93, 96, 97 boundary
94 false positive
95 false negative
100 Health advice screen
101 Clock-like dial
102, 103 Hand
111 safe area
111L, 111R Energizing electrode
112 need attention area
112L, 112R Measurement electrode
113 alert area
161L, 116R Energizing electrode
162L, 162R: Measurement electrode
501 Input section
502 Weight measurement section
503 Bioelectrical impedance measurement section
504 Memory section
505 Control section
506 Output section
510, 520, 530 Health level determination system

1. A health level determination system (510, 520, 530) comprising:
a bioelectrical impedance acquisition section (503, 53) that is configured to acquire one or more bioelectrical impedance of one or more body parts of a user; and
a health level determination section (505, 56, 56′, 56″) that is configured to determine a health level of the user based at least partially, indirectly or directly, on the bioelectrical impedance of the one or more body parts.

2. The health level determination system (510, 520) according to claim 1, wherein the health level determination section (505, 56, 56′, 56″) is configured to determine the health level of the user based at least partially, directly, on the bioelectrical impedance of the one or more body parts.

3. The health level determination system (510, 520) according to claim 1, further comprising a body composition estimation section (505, 54) that is configured to estimate a plurality of body compositions of the user from the bioelectrical impedances of the one or more body parts, wherein
the health level determination section (505, 56, 56′) is configured to determine the health level of the user based, at least partially, indirectly or directly, on the plurality of body compositions.

4. The health level determination system (510) according to claim 3, further comprising a medical checkup result estimation section (505, 55) that is configured to estimate a plurality of medical checkup results of the user from the plurality of body compositions, wherein
the health level determination section (505, 56) is configured to determine the health level of the user based at least partially on the plurality of medical checkup results.

5. The health level determination system (510) according to claim 4, wherein the medical checkup result estimation section (505, 55) is configured to estimate the plurality of medical checkup results of the user from the plurality of body compositions using an inference model.

6. The health level determination system (510) according to claim 4, wherein the health level determination section (505, 56) is configured to determine the health level of the user based at least partially on the plurality of medical checkup results using an inference model.

7. The health level determination system (510) according to claim 4, wherein each of the plurality of medical checkup results has a significant correlation with the plurality of body compositions.

8. The health level determination system (510) according to claim 5, wherein each of the plurality of medical checkup results has a significant correlation with the plurality of body compositions.

9. The health level determination system (510) according to claim 6, wherein each of the plurality of medical checkup results has a significant correlation with the plurality of body compositions.

10. The health level determination system (510, 520, 530) according to claim 1, wherein the health level determination section (505, 56, 56′, 56″) is configured to determine whether the user is healthy, unhealthy, or indeterminate.

11. The health level determination system (510, 520, 530) according to claim 2, wherein the health level determination section (505, 56, 56′, 56″) is configured to determine whether the user is healthy, unhealthy, or indeterminate.

12. The health level determination system (510, 520, 530) according to claim 4, wherein the health level determination section (505, 56, 56′, 56″) is configured to determine whether the user is healthy, unhealthy, or indeterminate.

13. The health level determination system (510, 520, 530) according to claim 7, wherein the health level determination section (505, 56, 56′, 56″) is configured to determine whether the user is healthy, unhealthy, or indeterminate.

14. The health level determination system (520) according to claim 10, further comprising an additional information acquisition section (505, 59) that is configured to request an input of additional information that was not used to determine the health level when the result of the determination is indeterminate, wherein
the health level determination section (505, 56′) is further configured to determine the health level using the additional information.

15. The health level determination system (520) according to claim 11, further comprising an additional information acquisition section (505, 59) that is configured to request an input of additional information that was not used to determine the health level when the result of the determination is indeterminate, wherein
the health level determination section (505, 56′) is further configured to determine the health level using the additional information.

16. The health level determination system (520) according to claim 12, further comprising an additional information acquisition section (505, 59) that is configured to request an input of additional information that was not used to determine the health level when the result of the determination is indeterminate, wherein
the health level determination section (505, 56′) is further configured to determine the health level using the additional information.

17. The health level determination system (510, 520, 530) according to claim 10, further comprising an output section (506, 58) that is configured to output the result of the determination of the health level, wherein
the output section (506, 58) is configured to display the result of the determination by pointing to the result of the determination on a clock-like graphic (101).

18. The health level determination system (510, 520, 530) according to claim 17, wherein the output section (506, 58) is configured to point to a predicted future determination result (103) on the clock-like graphic (101).

19. A health level determination program that, when executed by a processor, makes the processor (505) function as a health level determination section (56, 56′, 56″) that is configured to determines a health level of a user based, at least partially, indirectly or directly, on bioelectrical impedances obtained from one or more body parts of the user.

20. A health level determination server comprising a health level determination section (505, 56, 56′, 56″) that is configured to determine a health level of a user based, at least partially, indirectly or directly, on bioelectrical impedances obtained from one or more body parts of the user.
